(19) United States
(12) Patent Application Publication
(10) Pub. No.: US 2005/0129755 A1
(43) Pub. Date: Jun. 16, 2005

(54) Ingestible capsules containing ethanol

(76) Inventors: Paz Levy, Los Angeles, CA (US); Ido Lev, Los Angeles, CA (US)

Correspondence Address: KOPPEL, JACOBS, PATRICK & HEYBL, 555 ST. CHARLES DRIVE, SUITE 107, THOUSAND OAKS, CA, 91360, US

(21) Appl. No.: 10/738,243

(22) Filed: Dec. 16, 2003

(51) Int. Cl. A61K 47/00; A61K 9/48; A61K 9/64; A61K 31/045

(52) U.S. Cl. 424/451; 424/456; 426/592; 514/724

(57) ABSTRACT

An ingestible capsule containing substantially pure ethanol provided for medicinal or recreational purposes.

The present application relates to a method and devices for delivering ingestible ethyl alcohol, also referred to as ethanol, to an individual for recreational or medicinal purposes.

### FIELD OF THE INVENTION

Alcoholic beverages have been consumed by humans for centuries, generally for recreational purposes—more particularly to get a “buzz”, get high or get drunk. Beer is known to have been produced as early as 2000 BC, the Code of Hammurabi setting forth standards for beer production. While there are numerous different beverages which contain ethanol, typical alcoholic beverages are beer, wine and distilled spirits or “hard liquor”. Beer typically contains 3 to 6%ᵥ ethanol, usually around 5%ᵥ ethanol and wine typically contains about 9-14%ᵥ ethanol, typically about 12%ᵥ ethanol. Natural fermentation typically ceases at a 14%ᵥ ethanol content. However, the ethanol content can be increased by distillation to produce brandies, which may contain up to 20%ᵥ ethanol, or hard liquors. Hard liquor typically contains in excess of about 40%ᵥ ethanol (80 proof) which is produced by distillation. However, certain distilled spirits may have as much as 75%ᵥ ethanol (150 proof). Still further, with continued distillation substantially pure ethanol referred to as “grain alcohol” can be prepared. Because of the natural affinity of ethanol for water, and the difficulty of removing all of the water, the maximum concentration of grain alcohol is 95-97.5%, ethanol (190-195 proof).

A typical serving of each, namely 5 ounces of wine, 12 ounces of beer or 1.5 ounces of hard liquor (80 proof) delivers about the same amount of pure ethanol, namely 0.6 ounces or approximately 17.7 ml.

It has been recognized for some time that alcoholic beverages taken in moderation have a definite medicinal benefit. “Light-to-moderate ethanol consumption reduces the overall risk of stroke and the risk of ischemic stroke in men. The benefit is apparent with as little as one drink per week. Greater consumption, up to one drink per day, does not increase the observed benefit.” Possible mechanisms include an increase in high-density lipoprotein (HDL) cholesterol and a reduction in stress. (Berger K., et al “Light-to-moderate ethanol consumption and risk of stroke among U.S. male physicians”, N Engl J Med 1999;341,1557-64). This is supported by numerous other reports in medical journals. From epidemiological studies, primarily designed to identify factors associated with high mortality from coronary heart disease (CHD), it became apparent that these rates were lower among drinkers of small to moderate amounts of ethanol than among non-drinkers. (Yano, K., G. G. Rhoads and A. Kagan. “Coffee, ethanol and risk of coronary heart disease among Japanese men living in Hawaii.” New England J of Medicine. 297(1977), 405-9; Dyer, A. R., J. Stamler, O. Paul et al. “Ethanol consumption, cardiovascular risk factors, and mortality in two Chicago epidemiological studies.” Circulation. 56(1977), 1067-74; Marmot, M. G., M. J. Shipley, G. Rose and B. J. Thomas. “Ethanol and mortality: A U-shaped curve.” Lancet. 1(1981), 580-3; Gordon, T. and W. B. Kannel. “Drinking and mortality. The Framingham study.” American J. of Epidemiology. 120(1984), 97-107; Klatsky, A. L., M. A. Armstrong and G. D. Friedman. “Risk of cardiovascular mortality in ethanol drinkers, ex-drinkers and non-drinkers.” American J. of Cardiology. 66(1990), 1237-42; Boffetta, P. and L. Garfinkel. “Ethanol drinking and mortality among men enrolled in an American Cancer Society prospective study.” Epidemiology. 1(1990), 342-8; Doll, R. “Ethanol and health: An overview.” Conference: The Medicinal Virtues of Ethanol in Moderation. Sydney, Australia. Oct. 30-Nov. 1, (1991); de Labry, L. O., R. J. Glynn, M. R. Levenson, et al. “Ethanol consumption and mortality in an American male population: Recovering the U-shaped curve—Findings from the Normative Ageing Study.” J. of Studies on Ethanol. 53(1992), 25-32) and there was a general reduction in risk of major coronary events (Criqui, M. H. and B. L. Ringel. “Does diet or ethanol explain the French Paradox” The Lancet. 344(1994), 1719-23; Camargo, C. A., C. H. Hennekens, et al. “Prospective study of moderate ethanol consumption, aspirin therapy, and risk of myocardial infarction.” Amer J. of Epidemiology. Suppl 139(1994), Abstract 99; Muller, C. J. and K. C. Fugelsang. “Take two glasses of wine and see me in the morning.” The Lancet. 343(1994): 1428-9; Ridker, P. M., D. E. Vaughan, M. J. Stampfer, R. J. Glynn and C. H. Hennekens et al. “Association of moderate ethanol consumption and plasma concentration of endogenous tissue-type plasminogen activator. JAMA. 272(1994), 929-33; Klatsky, A. L. “Epidemiology of coronary heart disease—influence of ethanol.” Ethanolism: Clinical and Experimental Research. 18(1994): 88-97; Barrett, D., R. F. Anda, J. B. Croft, M. K. Serdula and M. J. Lane. “The association between ethanol use and health behaviours related to the risk of cardiovascular disease: the South Carolina Cardiovascular Disease Prevention Project.” J. of Studies on Ethanol. 56(1995): 9-15; Woodward, M. and H. Tunstall-Pedoe. “Ethanol consumption, diet, coronary risk factors, and prevalent coronary heart disease in men and women in the Scottish heart health study.” J. of Epidemiology and Community Health. 49(1995), 351-62; Saunders, J. B. “Ethanol as a benefit.” Drug and Ethanol Review. 14(1995), 3-6; Fuchs, C. S., M. J. Stampfer, G. A. Colditz, E. L. Giovannucci, J. E. Manson, I. Kawachi, D. J. Hunter, S. E. Hankinson, C. H. Hennekens, B. Rosner, F. E. Speizer and W. C. Willett. “Ethanol consumption and mortality among women.” The New England J. of Medicine. 332(1995), 1245-50; McElduff, P. and A. J. Dobson. “How much ethanol and how often? Population based case-control study of ethanol consumption and risk of a major coronary event.” British Medical J. 314(1997), 1159-64; Lazarus, R., D. Sparrow and S. T. Weiss. “Ethanol intake and insulin levels. The Normative Aging Study.” American J. of Epidemiology. 145(1997), 909-16; Wannamethee, G. S. and A. G. Shaper. “Lifelong teetotallers, ex-drinkers and drinkers: mortality and the incidence of major coronary heart disease events in middle-aged British men.” International J. of Epidemiology. 26(1997): 523-31).

Studies on ethanol and CHD led to other studies exploring ethanol and overall mortality rates (Duffy, J. C. “Ethanol consumption and all-cause mortality.” International J. of Epidemiology. 24(1995), 100-5; Doll, R., R. Peto, E. Hall, K. Wheatley and R. Gray. “Mortality in relation to consumption to ethanol: 13 years' observations on male British doctors.” British Medical J. 309(1994): 911-8) and other health issues. Today, the body of evidence strongly suggests that low to moderate consumption of beverage ethanol is, in fact, beneficial to health. This evidence is steadily growing, in scope and breadth.

The range of beneficial effects emanating from moderate consumption, based on just the most recent research, may be summarized as follows:
a protective effect with regard to ischaemic heart disease (IHD) (Serdula, M. K., S-L Koong, D. F. Williamson, R. F. Anda, J. H. Madans, J. C. Kleinman and T. Byers. “Ethanol intake and subsequent mortality: findings from NHANES I follow-up study.” J. of Studies on Ethanol. 56(1995), 233-239);
a protective effect with regard to cerebromuscular disease (Gronbaek, M., A. Deis, T. I. A. Sorensen, U. Becker, P. Schnohr and G. Jensen. “Mortality associated with moderate intakes of wine, beer, or spirits.” British Medical J. 310(1995), 1165-9)
protection against myocardial infarction (Marques-Vidal P., P. Ducimetiere, A. Evans, J-P Cambou and D. Arveiler. “Ethanol consumption and myocardial infarction: a case-control study in France and Northern Ireland.” American J. of Epidemiology. 143(1996), 1089-93; Hammar, N., A. Romelsjo and L. Alfredsson. “Ethanol consumption, drinking pattern and acute myocardial infarction. A case referent study based on the Swedish Twin Register.” J. of Internal Medicine. 241(1997), 125-31)
improved insulin sensitivity (Rimm, E. B., J. Chan, M. J. Stampfer, G. A. Colditz and W. Willett. “Prospective study of cigarette smoking, ethanol use, and the risk of diabetes in men.” British Medical J. 310(1995), 555-9; Kiechl, S., J. Willeit, W. Poewe, G. Egger, F. Oberhollenzer, M. Muggeo and E. Bonora. “Insulin sensitivity and regular ethanol consumption: large, prospective, cross sectional population study (Bruneck study).” British Medical J. 313(1996), 1040-4)
apparent reduction in peripheral arterial disease (PAD) (Jepson, R. G., F. G. R. Fowkes, P. T. Donnan and E. Housley. “Ethanol intake as a risk factor for peripheral arterial disease in the general population in the Edinburgh Artery Study.” European J. of Epidemiology. 11(1995), 9-14; Clevidence, B. A., M. E. Reichman, J. T. Judd, R. A. Muesing, A. Schatzkin, E. J. Schaefer, Z. Li, J. Jenner, C. C. Brown, M. Sunkin, W. S. Campbell and P. R. Taylor. “Effects of ethanol consumption on lipoproteins of premenopausal women: a controlled diet study.” Arteriosclerosis, Thrombosis and Vascular Biology. 15(1995), 179-84; Camargo, C. A., M. J. Stampfer, R. J. Glynn, J. M. Gaziano, J. E. Manson, S. Z. Goldhaber and C. H. Hennekens. “Prospective study of moderate ethanol consumption and risk of peripheral arterial disease in US male physicians.” Circulation. 95(1997), 577-80);
protection against Alzheimer's disease (Orgogozo, J-M., J-F. Dartigues, S. Lafont, L. Letenneur, D. Commenges, R. Salamon, S. Renaud and M. B. Breteler. “Wine consumption and dementia in the elderly: a prospective community study in the Bordeaux area.” Revue Neurologique. 153(1997), 185-192);
general reduction in the risk of coronary heart disease and major coronary events (Criqui and Ringel (1994) ibid.; Camargo, Hennekens et al (1994) ibid.; Muller and Fugelsang (1994) ibid.; Ridker et al (1994) ibid.; Klatsky (1994) ibid.; Barrett et al (1995) ibid; Woodward, Tunstall-Pedoe (1995) ibid.; Saunders (1995) ibid.; Fuchs et al (1995) ibid.; McElduff et al (1997) ibid.; Lazarus et al (1997) ibid.; Wannamethee and Shaper (1997) ibid.;
significantly decrease all causes of mortality (Duffy (1995) ibid.; Doll et al (1994) ibid.);
lower prevalence of a range of chronic diseases (La Vecchia, C., A. Decarli, S. Franceschi, M. Ferraroni and R. Pagano “Prevalence of chronic diseases in ethanol abstainers.” Epidemiology. 6(1995), 436-8.);
serving as a proxy for a spectrum of generally moderate behaviors that either attenuate the effect of stress on depression or suppress the effects of stress (Lipton, R. I. “The effect of moderate ethanol use on the relationship between stress and depression.” American J. of Public Health. 84(1994), 1913-7.);
serving as a digestive aid (Weisse, M. E., B. Eberly and D. A. Person. “Wine as a digestive aid: comparative antimicrobial effects of bismuth salicylate and red and white wine.” British Medical J. 311(1995), 1657-60).

U.S. Pat. No. 4,507,327 to Ueda discloses a process for encapsulating foods and drinks, such as alcoholic beverages. The process first forms water filled alginate capsules. These capsules are immersed in the desired alcoholic beverage, such as wine, resulting in at least some of the water in the capsule being replaced by wine. Because of the significant solubility of water in any alcoholic beverage, the result is an alginate capsule containing a highly diluted ethanol-containing liquid. The intended use of the capsule appears to be for addition to other food stuffs and not directly swallowed. In addition, ethanol has been used as a solvent or carrier for pharmaceuticals enclosed in capsules. But there has been no suggestion of the delivery of capsules containing substantially pure ethanol for medicinal purposes.

### BRIEF DESCRIPTION

A convenient and unobtrusive method of delivering ethanol, for either its recreational or medicinal uses, comprises providing a capsule of a size suitable for swallowing, wherein the capsule contains an alcoholic solution of a high ethanol content, preferably 190 proof or greater ethanol.

### DETAILED DESCRIPTION

The present invention comprises the preparation of ingestible capsules containing ethanol, preferably 190 proof ethanol. These capsules are of a size suitable for swallowing whole, in the same manner as a pill, with the contents being released in the stomach or intestines at a time dependent on the dissolution rate of the capsule material.

Capsules of various standard dimensions and capacities, made from various materials and capable of enclosing solid materials, powders or liquids are generally available to the pharmaceutical and nutraceutical industry. Standard size and designations of capsules are listed in Table 1. However, larger sizes can be used. For example, a capsule the size of a newly developed ingestible camera used for intestinal tract exploration, which is approximately 30 mm by 11 mm, can be readily swallowed. Such a capsule would have a capacity of between 2 and 3 ml. Capsules with a capacity of 3 to 5 ml are available but are not used for human ingestion because their larger size makes them to more difficult to swallow. However, one skilled in the art will recognize that it is not necessary for an individual to swallow one large capsule, the same effect being accomplished by ingesting several smaller capsules at one time or over a period of time.

TABLE 1

| | VOLUME | DIMENSIONS |
|---|---|---|
| CAPSULE SIZE | (ml) | (mm) |
| 000 | 1.37 | |
| 00 | 0.95 | 20.22 × 8.18 |
| 0 | 0.68 | 18.44 × 7.34 |
| 1 | 0.50 | 16.61 × 6.63 |
| 2 | 0.37 | |
| 3 | 0.30 | 13.59 × 5.57 |
| 4 | 0.20 | |
| 5 | 0.13 | |

Typical materials of construction include either hard or soft gelatin or cellulosic materials such as hydroxypropyl methyl cellulose (HPMC). However, use of other suitable materials is not excluded. For quick release of its contents gelatin capsules are preferred. They are generally not dissolved by ethanol. For time release of the contents HPMC capsules are preferred because they can be designed to have a swell rate once ingested which can be utilized to provide a slow but continuous release of their contents. Both are suitable for containing liquids and can be readily sealed and made leak proof.

A preferred ethanol delivery capsule comprises a 000 gelatin capsule containing about 1.37 ml of ethanol, preferably 150 to 190 proof ethanol, designed to release its contents within about 2-30 minutes after ingestion. 13 capsules of 190 proof ethanol would provide an amount of ethanol equivalent to one 5 oz glass of wine, a 12 oz mug of beer or a shot of whiskey. An equivalent amount of ethanol would be provided by ingesting 6 capsules, each with a 3 mm capacity. As an alternative, to provide a relatively constant delivery of the same amount of ethanol aver a 24 hour period, multiple 000 ethanol capsules could be ingested at preset time periods, for example every 2 hours for an 18 or 24 hour period, 18 hours generally representing the non-sleeping portion of a 24 hour period.

One skilled in the art will recognize that while the largest size, readily ingestible capsule is preferred, the same desired end result can be accomplished by providing multiple smaller capsules. It is also contemplated that lower proof alcohol capsules, for example, 80 proof can be provided but then more capsules will be required to obtain the same dosage. As an example, the capsule could contain standard beverage such as gin, vodka, scotch, whiskey, etc.

1. A method of delivering ethanol to an individual comprising providing one or more ingestible capsules containing a liquid solution consisting essential of ethanol and a diluent.

2. The method of claim 1 wherein the capsule is formed from an ethanol impermeable gelatin or hydroxypropyl methyl cellulose.

3. The method of claim 2 wherein the capsule has a capacity of at least about 1.3 ml.

4. The method of claim 2 wherein the liquid solution is at least about 150 proof ethanol.

5. The method of claim 2 wherein the capsule is filled with 190 proof ethanol.

6. The method of claim 1 wherein sufficient capsules are ingested within a defined period of time by an individual to deliver a total of about 17 ml of ethanol.

7. A method of preventing or retarding ischemic heart disease or cerebromuscular disease, preventing myocardial infarction, improving insulin sensitivity, reducing peripheral arterial disease, protecting against Alzheimer's disease, reducing the risk of coronary heart disease, reducing the effect of stress or aiding digestion comprising delivering an oral dosage, on a daily basis of a therapeutically effective amount of ethanol, the ethanol being enclosed in an ingestible capsule which releases its contents in the stomach or intestinal tract.

8. The method of claim 7 wherein the capsule is formed from an ethanol impermeable gelatin or hydroxypropyl methyl cellulose, the ethanol is at least about 150 proof and the therapeutically effective amount is at least about 17 ml per 24 hour period.

9. An ethanol impermeable gelatin or hydroxypropyl methyl cellulose capsule the contents thereof consisting essentially of a solution of ethanol, said capsule having exterior dimension suitable for a human to swallow.

10. The capsule of claim 9 having a capacity of from about 0.13 to about 3 ml.

11. The capsule of claim 9 having a capacity of at least about 1.3 ml.

12. The capsule of claim 9 containing at least about 150 proof ethanol.

13. The capsule of claim 9 containing at least about 190 proof ethanol.
